(19) United States
(12) Patent Application Publication
(10) Pub. No.: US 2009/0227035 A1
(43) Pub. Date: Sep. 10, 2009

(54) METHOD AND TEST KIT FOR THE DETERMINATION OF IRON CONTENT OF IN-USE LUBRICANTS

(75) Inventors: Beatrice I. Naudts, Lochristi (BE); Mark G. Proot, Torhout (BE)

Correspondence Address: CHEVRON CORPORATION, P.O. BOX 6006, SAN RAMON, CA, 94583-0806, US

(73) Assignee: Taxaco Development Corporation

(21) Appl. No.: 12/405,881

(22) Filed: Mar. 17, 2009

Related U.S. Application Data
(63) Continuation-in-part of application No. 11/137,249, filed on May 24, 2005, now abandoned.

(51) Int. Cl. G01N 33/28; G01N 21/75; B01L 11/00

(52) U.S. Cl. 436/60; 422/61

(57) ABSTRACT

The present invention relates to a method to determine the iron content of in-use lubricants by means of visible spectroscopy. The invention further relates to a test kit using the method. The test may be conducted on-site. The method of the present invention provides an accurate alternative to the standard Plasma-Atomic-Emission spectroscopic technique (ICP-AES), which must be done in a laboratory. One of the application areas is iron measurement of in-use lubricants having a Base Number up to 75 which are possibly contaminated to a high extent with soot, ash, metal debris etc.

### CROSS-REFERENCE TO RELATED APPLICATIONS

This application is a continuation-in-part of copending U.S. patent application Ser. No. 11/137,249 and claims priority therefrom.

### FIELD OF THE INVENTION

The present invention relates to a method of preparing an in-use lubricant for determination of the wear metal content by means of visible spectroscopy. The present invention is also directed to a test kit using the method, which may be conducted on-site.

### BACKGROUND OF THE INVENTION

Oil that is used for lubricating machines or engines, i.e., lubricant, is subject to degradation, chemical decomposition and depletion of essential additives. Machines or engines themselves have the effect of causing a build-up of contaminant materials within the lubricant. Therefore the lubricant must be periodically monitored in order to determine its condition and its concentration of contaminant materials. As the contaminant material concentration increases, the remaining usable lifetime of the oil decreases to the point where continued use of oil containing a high concentration of contaminants is detrimental to the proper operation of the machine or engine. Such contaminant concentration increase necessitates replacement of the oil with oil containing a low concentration of contaminant materials to avoid machine or engine damage. Conversely, changing the lubricant too early in its operational lifetime results in significant and unnecessary expense.

Oil analysis is a commonly accepted form of predictive maintenance technology. It can be applied to equipment utilization, maintenance and management. The nature and concentration of the contaminants may reveal faulty operator practices.

Machines, engines, or compressors used to power oil drilling, petrochemical or transportation equipment, utilize many gallons of lubricant. It is standard practice of oil manufacturers to add to the oil active extreme pressure and/or anti-corrosive materials which tend to inhibit the formation and/or build-up of contaminant materials. As the machines, engines, or compressors are operated, the concentration of the additives is depleted to the point where they fail to perform their inhibitory function, thus resulting in discernible increases in the amount of contaminant material existing within the lubricant. Further, these additives may be organic and/or organo-metallic chemical compounds which, due to the operating environment and conditions of the machines, may degrade into acidic and/or basic components. Such acidic and/or basic materials may have detrimental effects on the internal components of the machines, thereby also necessitating oil replacement.

Characteristically, the lubricant used in drilling, petrochemical, or transportation machines or engines has an operational lifetime dependent upon the quality of the lubricant, method of operation of the machines or engines, possible process contamination inherent in petrochemical production, and the environment parameters to which the lubricant is subjected. Failure to replace lubricant that contains a high concentration of contaminant material causes damage to the machines or engines themselves and results in very significant repair and replacement costs. Many prior methods for determining the concentration of contaminant materials in lubricants as generally described in chemical texts and ASTM manuals have utilized chemical procedures performed by a chemist or highly skilled technician at a laboratory site, all at a great cost of time and money.

The current methods of analyzing lubricants require that samples be sent to laboratories relatively far removed from the operational site. Since machines or engines are, in numerous industrial operations, used continuously it is essential that information regarding the quality of the lubricant be transmitted to the industrial site as quickly as possible to avoid the possibility that the lubricant then in use within the machines has exceeded its useful lifetime. As often is the case, current laboratory analysis of the lubricant occurs at a place relatively far removed from the industrial site, which consumes valuable time often in excess of the critical periods during which damage to the machines can occur.

This delay is due to the time involved in withdrawing a sample of the lubricant, sending it to a laboratory, analyzing the sample, and transmitting the results back to the industrial site. Because of this, a common practice in industries such as the well drilling, petrochemical, and transportation industries is to replace the lubricant after an established operational lifetime dependent upon the operational and environmental parameters existing at the industrial site and, in some instances, without regard to the concentration of contaminants. A drawback of this practice is that very often the lubricant is replaced before the contaminant concentration is sufficiently high to warrant such replacement, which adds an unnecessary operating expense. Among other things, the use of a method or test kit, which correlates well with standard laboratory equipment, in accordance with the present invention overcomes this drawback.

The method and test kit of the present invention are applicable to a great variety of in-use lubricants having a Base Number up to 75 even when such fluids are highly contaminated with soot, ash, metal debris etc. This present invention was developed with and is most preferably used for the on-site analysis of cylinder drip oils. As discussed more fully herein, having an on-site test kit enables an engineer to quickly evaluate the wear rate of each cylinder of low speed diesel engines and to act accordingly by optimizing the feed rate of the lubricant. Such a prompt action is vital to maintaining satisfactory engine performance and to avoid the severe problems associated with engine wear, which result in huge expense.

The description and background discussion of the present invention use cylinder drip oils as a primary example because such drip oils represent a great diversity of lubricants, which vary greatly with respect to Base Number (BN-value) and contaminant level. Cylinder drip oil is unburnt oil of two stroke large diesel engines that is scraped down the cylinder liner walls during each stroke of the engine.

The rate and extent of ring and liner wear in low speed marine diesel engines depends greatly on the lubricating process used for the engine. The elements of the lubricating process are the applied lubricant, the engine design, the operating conditions and the maintenance.

The ring and liner wear is produced from a combination of corrosive, adhesive and abrasive wear. Under normal operating conditions, corrosive wear contributes most to the total wear of the liner. The corrosive wear is the result of a chemical reaction between the ring/liner material and the sulfuric acids originating from the fuel combustion gases.

It is promoted by the condensation of (sulfuric) acids on the liner. To reduce the corrosive wear the condensation of the acids should be minimized and the ring and liner surface should receive maximum protection by the lubricant. Corrosive wear will increase the iron content of the drip oil. For this reason the iron content is a parameter to monitor the corrosive wear process in the engine. It is also a key indicator for a protective lubrication management and overhaul planning. A significant advantage of on-site testing of engine lubricant is that lubricant feed rate adjustment decisions can be made quickly in order to avoid the risk of engine damage.

Corrosive wear is generally considered acceptable if the iron content of the drip oil remains below 150 ppm. The level of iron found back in the drip oil when an engine is running at optimum oil feed rate will however vary from engine to engine and is dependent on the operating conditions. Increasing corrosive wear is taking place in the engine when the iron content of the drip oil rises above the 150 ppm level. Iron levels higher than 400 ppm indicate problems in the engine as a result of extreme wear. Therefore, to be most effective for this application an on-site test kit must be capable of a good assessment of the iron content of the drip oil up to 400 ppm.

As previously noted, corrosive wear will increase the iron content of cylinder drip oil. For this reason the iron content is a parameter (as well as the Base Number (BN value)) to monitor the corrosive wear process in the engine. It is also a key indicator for a protective lubrication management and overhaul planning.

ExxonMobil introduced the Scrape Down Analyser, a portable oil tester that measures the amount of iron (wear metal) in the drip oil. The analyzer is based on Kittiwake Analex PQ magnetism technology. The PQ operates by measuring the distortion of a magnetic flux field when a ferromagnetic sample is placed in the field.

Midas Tester (Manor Technology Monitoring Ltd) is a portable analyser for the determination of iron in lubricants. It is based on a principle similar to the abovementioned Exxon Mobil Analyser. A small container with the lubricant sample is placed in the instrument and iron content is directly displayed in ppm units.

U.S. Pat. No. 4,203,725 relates to an on-site method for determining the need for replacement of oil due to the build up of metallic contaminant material therein, which involves vigorously mixing a known volume of oil in a known volume of an aqueous solvent which extracts metal and/or metal oxides existing within the oil from the oil into the aqueous solvent including a reagent capable of reacting with the metal and/or metal oxide and comparing the color as generated in the aqueous phase by the reaction of said reagent with said metal and/or metal oxide to a standard color to determine the concentration of said metallic contaminant material.

U.S. Pat. No. 5,194,910 relates to methods and apparatus employing optical spectrometry techniques to measure metallic wear debris contamination of used motor oil.

U.S. Pat. No. 4,238,197 (Eisentraut et al) discloses a test method for the analysis of lubricating oils for iron content, which involves a more complex series of steps then that set forth in the current invention. Furthermore in Eisentraut et al, reagents are added individually to the oil to be tested. In the instant invention, an in-use lubricant is added to a solvent comprised of all of the reagents. This single addition step greatly enhances ease of use.

### SUMMARY OF THE INVENTION

The present invention relates to a method for preparing in-use lubricant for determination of its wear metal content by photometric means, wherein such preparation and determination may be used on-site, said method comprising the following steps:
(a) adding a predetermined amount of said in-use lubricant to an active solvent comprising predetermined amounts of an apolar organic solvent, a polar organic solvent, an organic acid, water, an iron complexing agent and a reducing agent, said active solvent having a pH between 2 and 4;
(b) thoroughly mixing the lubricant and the active solvent until the lubricant is completely dissolved;
(c) allowing the mixture to react completely and to separate into a top layer and a bottom layer, the bottom layer comprising an iron complex;
(d) filtering at least a portion of the bottom layer of the mixture directly into a receptacle suitable for absorbance measurement in the visible range.

### BRIEF DESCRIPTION OF THE DRAWINGS

FIG. 1 is VIS spectrum of a series of drip oil samples having up to 400 ppm iron content.

FIG. 2 is a calibration curve used to convert absorbance measurements to iron content.

FIG. 3 is a graph and histogram comparing the results of the invention and ICP-AES methods

FIG. 4 is a pictorial representation of a test kit and method according to the present invention.

FIG. 5 is a pictorial representation of an alternative embodiment of a container and filter according to the present invention.

### DETAILED DESCRIPTION OF THE INVENTION

The present invention relates to a method to determine the iron content of in-use lubricants by means of visible spectroscopy and to a test kit which uses the method, which may be conducted on-site. The method of the present invention provides an accurate alternative to the standard Plasma-Atomic-Emission spectroscopic technique (ICP-AES), which must be done in a laboratory.

The present invention is derived from the development of a method and test kit by visible spectroscopy for the determination of the iron content of in-use lubricants, for example, cylinder drip oils. The method according to the invention gives an accurate assessment of the iron content up to 400 ppm and an indication when the iron content is above that level. The method is applicable to a great diversity of lubricants. These differ in terms of lubricant technology, Base Number (that can range up to 75 BN), and type and amount of contamination (e.g. soot, ash, metal debris, water etc.).

One advantage of the present invention is that a test kit made in accordance with the invention can be used on-site as a good alternative for the reference ICP-AES analysis technique. ICP-AES is a standard tool in Used Oil Analysis Programs that allows the detection of abnormal wear in a system because of its efficiency to detect dissolved or pseudo dissolved metallic forms in the lubricant up to a particle size of 5 μm.

It has been found that the method of this present invention gives unbiased test results in comparison with the standard ICP-AES method for an iron content range up to 400 ppm. This gives also the advantage that the normal interpretation for the monitoring process of two stroke large diesel engines, which is routinely based on ICP-AES test results, can be maintained.

Another advantage of the present invention is the incorporation of the method into a test kit, which may be used on-site. Despite the chemical complexity of the test, the equipment and procedure are kept simple and do not require special skills from the operator. A dedicated spectrometer may be designed exclusively for the purpose of carrying out the method of the present invention, which would be used to measure light absorbing properties of the test solutions and to display these measurements directly as ppm iron content.

Another advantage of a test kit made in accordance with the present invention is that the consumable units may be designed in such a way that a robust and accurate test kit is obtained which requires only limited intervention of the operator.

### The Complexing Agent

Among other factors, a method according to the present invention is based on the formation of a coloured complex between the iron present in the lubricant and a complexing agent that turns with iron ions to colour of which the intensity is measured.

Colour systems for the detection of iron are described for example in U.S. Pat. No. 5,763,281 and references cited therein. Complexing agents of the ferroin type which yield a dye with iron that can be assayed photometrically may be suitable. Such substances are for example bathophenanthroline, ferene (3-(2-pyridyl)-5,6-bis(2-[furylsulfonic acid])-1,2,4-triazine disodium salt) and ferrozine (3′(2′-pyridyl)-5,6-diphenyl-1,2,4-triazine-sulfonic acid disodium salt). The complexing agent should be selected so that the dye formation is proportional to the iron content of the sample and as a consequence can be assayed photometrically.

A preferred complexing agent is Ferrotrace, a product of Mistral Detection Ltd. that is extremely sensitive and selective to trace amounts of iron and turns iron ions (as Fe²⁺) to a dark violet colour.

### The Solvent Medium

In order to allow the formation of the iron complex and its spectroscopic measurement, lubricants such as cylinder drip oil samples are diluted and treated with an appropriate medium. The medium, sometimes herein referred to as an “active solvent”, comprises a neutral solvent, a complexing agent and a reducing agent. The lubricant sample diluted with active solvent is sometimes herein referred to as the “test solution”.

Another aspect of the present invention is the nature of the active solvent. An active solvent in accordance with the present invention will have multifunctional properties to enable it to be used for the analysis of a great diversity of lubricant samples. In addition to isolating, eventually reducing, and complexing iron ions present in the lubricant, the active solvent provides the optimum environment to minimise potential interference with these processes due to the presence of contaminants (e.g. soot, ash etc) and compounds (like detergents) in the lubricant. The active solvent is composed in a way that the absorbance measurements are selective for iron and are not affected by the type and state of the lubricant. This results in a consistent Lambda max at the appropriate frequency, for example, 550 nm.

An active solvent according to the present invention will most preferably have the following combination of properties:
(a) Be chemically inert with respect to the complexing agent;
(b) Have a Lambda cut-off below 350 nm;
(c) Be both a good solvent for the lubricant and for the iron complexing agent/complex. It will provide a good mixing between the apolar and polar components present in the test solution, but when the solution is standing, ensure a very quick separation into an apolar layer (top), containing lubricant and contaminants and polar (bottom) layer, the latter of which contains the coloured iron complex;
(d) Establish a constant ratio between the apolar layer and the polar layer of the test solution for any lubricant;
(e) Contain an amount of acid effective: 1) to neutralize the detergents present in any lubricant so as to eliminate the negative effect of detergents on the formation and detection of the coloured complex and 2) to have a pH of the test solution between 2 and 4 in order to develop the correct colour of the complex; and
(f) Be stable for an extended period of time when mixed with the complexing agent.
(g) Be environmentally friendly and safe.

The neutral solvent used in accordance with the invention is tailored to fulfil the objectives as set forth above. The neutral solvent comprises a mixture of from 20 to 30 vol % apolar organic solvent from 45 to 55 vol % polar organic solvent, from 5 to 15 vol % of a low molecular weight organic acid and from 10 to 20 vol % water, preferably deionised water.

Examples of apolar organic solvents include: iso and normal alkanes from C6 through C16; ethers (ROR′) where R and R′ are a combination of C1 through C4. Preferably n-octane is used.

Examples of polar organic solvents include: acetonitrile; acetone; alcohols from C2 through C6; dimethylsulfoxide. Preferably acetone is used.

Examples of low molecular weight organic acids include: acetic acid; propionic acid. Preferably, acetic acid is used.

### The Test Kit

The present invention provides a lubricant analysis test kit for determining the iron content of an in-use lubricant, which is designed to be used on-site, as an alternative to the standard Plasma-Atomic-Emission spectroscopic technique (ICP-AES).

A kit in accordance with the invention includes receptacle adapted to be opened and closed on one end and being further adapted to prepare a test solution containing the lubricant. A flexible transparent or semi-transparent plastic container with a screw cap is preferred.

The receptacle also contains at least two crushable ampoules which together contain all the components of the active solvent. In a preferred embodiment, four crushable ampoules are used. One of the ampoules contains a predetermined amount of an apolar organic solvent. The second ampoule contains a predetermined amount of a polar organic solvent and an iron complexing agent. The third ampoule contains a predetermined amount of an organic acid and water. The fourth ampoule contains a reducing agent;

The ampoules are preferably made from thin glass. The nature and amount of the solvents and other chemicals in the ampoules is as discussed herein.

It is also possible to use, for example, two crushable ampoules and a reducing agent in solid form, such as a pill. In this case, one of the ampoules might contain the apolar organic solvent and the other would contain the polar organic solvent, organic acid, water and iron complexing agent. Other combinations are possible and will be apparent to a skilled artisan.

A kit in accordance with the invention includes a dispensing device adapted to dispense a predetermined amount of lubricant into the receptacle. One such dispensing device is a micropipette (Transferpettor) of e.g. 100 μl for sampling the lubricant.

A kit in accordance with the present invention also includes filtration means to filter fluid simultaneously as said fluid is removed from the receptacle The filtration means is adapted to be fitted to the end of the receptacle when the end is open. A disc filter (e.g. in PTFE) of 0.45 μm pore diameter, used to filter the test solution prepared in the receptacle is a suitable example.

A kit in accordance with the present invention includes a second receptacle which is adapted to contain fluid and to be placed in a means to measure absorbance, for example, a differential spectrometer. For example, a VIS cuvette with a path length ranging between 0.2 cm and 1.0 cm, and having a volume of about 1 ml could be used. An Eppendorf cuvette with a path length of 0.2 cm and a small volume is preferred.

A test kit in accordance with the present invention includes means to measure the absorbance at a selected frequency and a baseline frequency and to translate and display these measurements as ppm iron content. An example is a differential spectrometer, which is able to measure the absorbance readings at, for example, 550 nm and 671 nm, the latter as baseline, and to translate and display these measurements as ppm iron content. This conversion can be established via the software of the instrument. Other instruments used to measure optical absorbance in the visible spectrum are well known and available to the person of ordinary skill in the art.

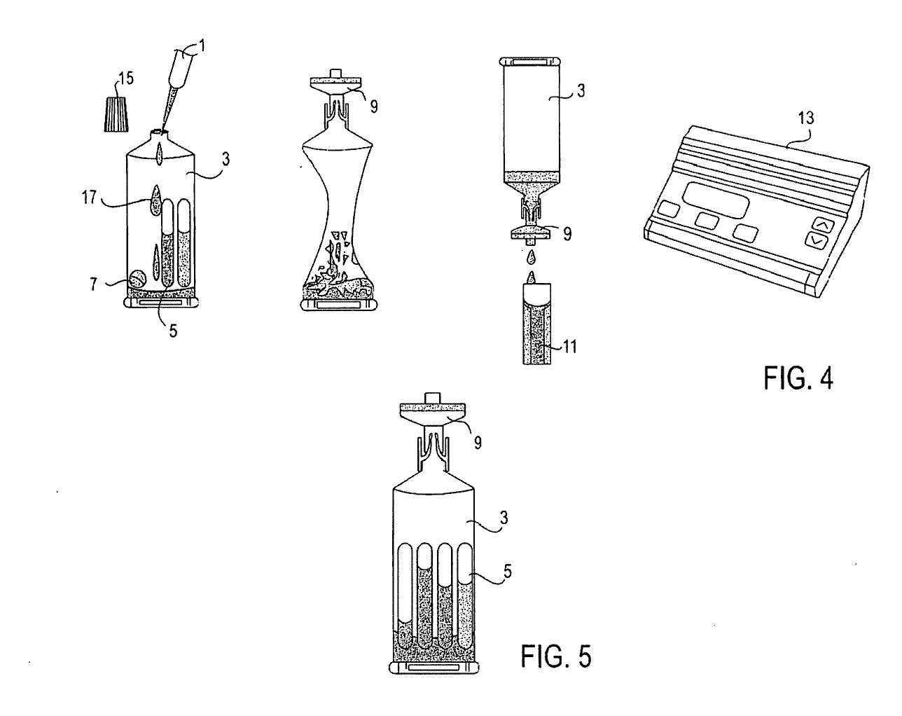

For example and with reference to FIGS. 4 and 5, a test kit may comprise:
(a) A Differential Spectrometer 13. The instrument is able to measure the absorbance readings at 550 nm and 671 nm, the latter as baseline, and to translate and display these measurements as ppm iron content. This conversion can be established via the software of the instrument.
(b) A micropipette 1 (Transferpettor) of e.g. 100 μl for sampling the lubricant.
(c) Consumables where one unit includes: a flexible transparent or semi-transparent plastic container 3 containing four crushable ampoules 5 (FIG. 5) or two crushable ampoules 5 and a solid reducing agent pill 7 (FIG. 4); a disc filter 9 and a VIS cuvette 11.
   (i) Plastic container 3 closes with a screw top 15, which can be replaced by an adapter with a disc filter 9.
   (ii) One crushable ampoule 5 contains the apolar organic solvent, 0.90 ml n-octane. One crushable ampoule 5 contains the polar organic solvent, 1.80 ml acetone and the iron complexing agent. One crushable ampoule 5 contains the organic acid and deionised water, in total 0.90 ml being a mixture of 40.00 vol % acetic acid and 60.00 vol % water. One crushable ampoule 5 contains a reducing agent, such as ascorbic acid. (FIG. 5)
(iii) A disc filter 9 (e.g. in PTFE) of 0.45 μm pore diameter, used to filter the test solution prepared in (1).
(iv) VIS cuvette 11 with a path length ranging between 0.2 cm and 1.0 cm, and having a volume of about 1 ml. In our case an Eppendorf cuvette with a path length of 0.2 cm and a small volume is used.

The following is an outline description of a method of using a test kit according to the invention with reference to FIG. 4.

A predetermined amount of lubricant sample 17 is added to a specifically devised container 3 that opens/closes with a screw top 15 and that contains a predetermined amount of active solvent stored in crushable ampoules 5 and optionally a solid reducing agent in the form of a pill 7. The ampoules 5 are crushed. The whole content of the container is then mixed and shaken until the lubricant is completely dissolved. The test solution is allowed to stand for a certain period to allow the test solution mixture to react completely (from one to three hours depending on the soot content of the sample being lower/higher than 1% m/m respectively). During this time it is preferred to place the container in a horizontal position in order to increase the contact surface between the two layers. The screw top 15 of the container 3 is then replaced by a disc filter 9 and the test solution is allowed to separate into a top layer and a bottom layer, which takes a few seconds. All interfering components such as soot, additives, contaminants and the oily medium are in the top layer. Importantly, the bottom layer contains the iron complex. At least a portion of the bottom layer of the test solution is pushed through the filter directly into a cuvette for VIS measurements.

The instrument used for the development of the subject method and the validation of the on-site test kit is a UV-VIS spectrometer (Type PerkinElmer Lambda Bio 40) with a scanning range of 200 to 900 nm.

With the lab UV-VIS instrument, the absorbance reading measured at 550 nm with 800 nm as baseline, is proportional to the iron content of the lubricant. To convert absorbance readings to iron content, a calibration curve is established using test solutions prepared with lubricant samples of known iron content as determined by ICP-AES. The obtained calibration curve allows calculating the iron content of blind lubricants expressed in ppm units.

With the on-site spectrometer, the absorbance reading measured at 550 nm with 671 nm as baseline, is proportional to the iron content of the lubricant. To convert absorbance readings to ppm iron content of the lubricant, an empirical factor is used. That factor is based on the calibration outcome of the lab UV-VIS instrument and a determined correlation factor that exists between the lab UV-VIS instrument and the on-site spectrometer. Specific filters can also be used to calibrate and verify the on-site spectrometer.

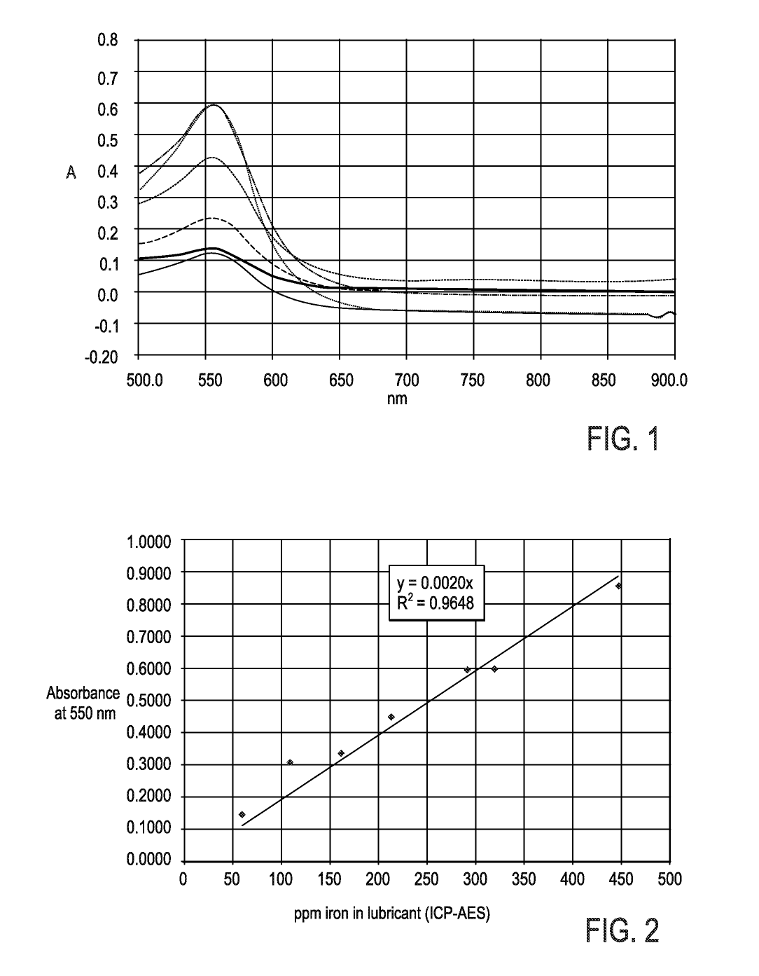

FIG. 1 shows the VIS spectrum obtained after treatment with the active solvent for a series of lubricant samples using lab equipment. The absorbance peak situated at 550 nm represents the iron complex formed. Its peak height with baseline at 800 nm is proportional to the iron content of the lubricant. A baseline correction is necessary because, dependent on the type of sample, the test solution is not always 100% clear after filtration.

### Calibration Curve

An example of a typical calibration curve is given in FIG. 2 while its precision is illustrated in Table I. A satisfactory linear calibration curve through zero is obtained up to 450 ppm iron in the lubricant. Note that the differences found between the calculated and target values are within the reproducibility constraint of the ICP-AES method.

TABLE I
Calibration Curve Data
ppm iron

| | VIS | |
|---|---|---|
| ICP | calculated | difference |
| 58 | 73 | 15 |
| 91 | 88 | −3 |
| 108 | 154 | 46 |
| 162 | 169 | 7 |
| 213 | 224 | 11 |
| 447 | 427 | −20 |

### Testing the Stability of the Coloured Iron Complex

When combining the active solvent with the lubricant sample, it takes some time to develop the coloured iron complex (typical dwelling time is set to from one to three hours depending on the soot content of the sample being lower/higher than 1% m/m respectively). To check the stability of the coloured complex, the filtered test solution was measured again after 1 day of storage. The repeatable test results, as summarized in Table II, show that the intensity of the colour remains constant for quite some hours which is a necessity for having a robust method.

TABLE II
Testing the Stability of the Coloured Iron Complex

| | | | | ppm iron | | |
|---|---|---|---|---|---|---|
| interval | | | BN | | VIS | |
| days | wo | type | value | ICP | calculated | difference |
| 0 | 70082 | Taro S. | 22.3 | 334 | 295 | −39 |
| 1 | | | | | 290 | −44 |
| 0 | 132233 | Taro S. | 30.4 | 127 | 126 | −1 |
| 1 | | | | | 127 | 0 |

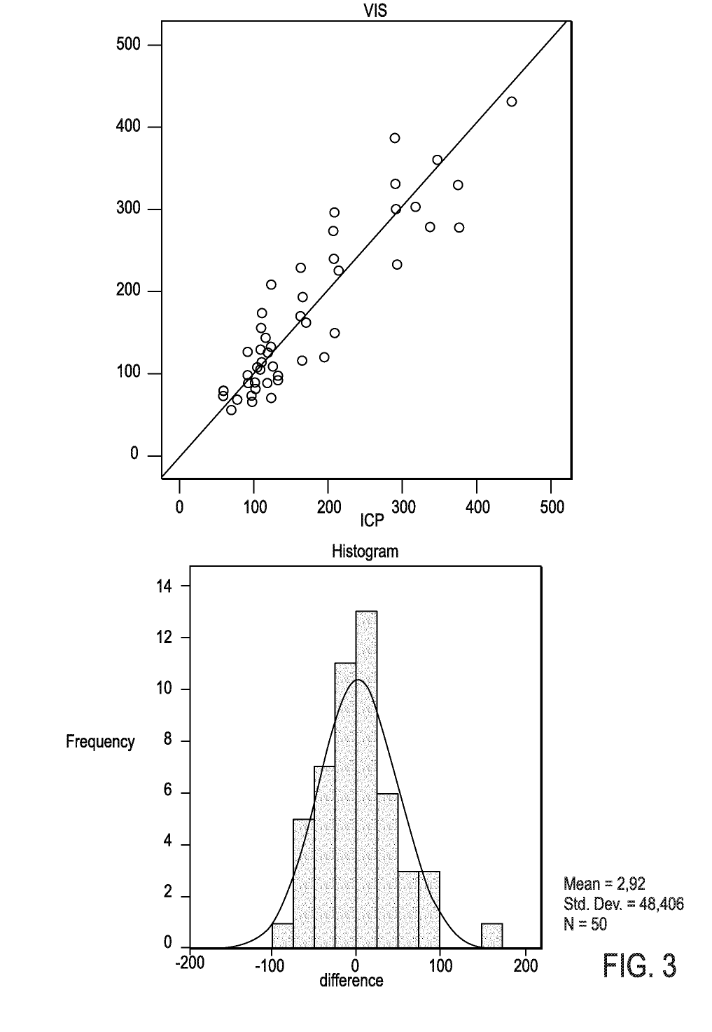

A series of lubricant samples of different iron content, as determined by ICP-AES, were tested according to the subject method. The obtained test results are shown graphically in FIG. 3. They show that there is a satisfactory linear relationship between the outcome by the method according to the present invention and ICP-AES reference method for an iron content range from zero to 400 ppm. The histogram of FIG. 3 further shows that the differences found between the subject method and the ICP-AES reference method are acceptable when compared to the test precision of the reference method.

Statistical analysis further demonstrates that the present method gives unbiased test results towards the ICP-AES method. This means that the method of this invention has the potential to be an acceptable alternative to the reference standard method.

There are numerous variations on the present invention which are possible in light of the teachings and supporting examples described herein. It is therefore understood that within the scope of the following claims, the invention may be practiced otherwise than as specifically described or exemplified herein.

What is claimed is:

1. A method for preparing in-use lubricant for determination of its wear metal content by photometric means, wherein such preparation and determination may be used on-site, said method comprising the following steps:
(a) adding a predetermined amount of said in-use lubricant to an active solvent comprising predetermined amounts of an apolar organic solvent, a polar organic solvent, an organic acid, water, an iron complexing agent and a reducing agent, said active solvent having a pH between 2 and 4;
(b) thoroughly mixing the lubricant and the active solvent until the lubricant is completely dissolved;
(c) allowing the mixture to react completely and to separate into a top layer and a bottom layer, the bottom layer comprising an iron complex;
(d) filtering at least a portion of the bottom layer of the mixture directly into a receptacle suitable for absorbance measurement in the visible range.

2. A method according to claim 1, wherein photometric means further comprises:
(a) photometrically measuring the net absorbance of the filtered solution at frequencies in the visible range; and
(b) converting the absorbance measurement to ppm iron content of the lubricant.

3. The method of claim 1, wherein the wear metal is iron.

4. A method according to claim 1, wherein the active solvent comprises from 20 to 30 vol % apolar organic solvent, from 45 to 55 vol % polar organic solvent, from 5 to 15 vol % of a low molecular weight organic acid and from 10 to 20 vol % water.

5. A method according to claim 1, wherein the reducing agent is in solid form.

6. A method according to claim 2, wherein the net absorbance is measured at a frequency between 525 nm and 575 nm with a fixed baseline between 650 nm and 900 nm.

7. A method according to claim 1, where the lubricant has a BN value of up to 75.

8. A method for determining the iron content of an in-use lubricant, which may be conducted on-site comprising:
(a) adding a predetermined amount of an in-use lubricant to a receptacle adapted to be opened and closed on one end and being further adapted to prepare a test solution containing the lubricant said receptacle containing therein at least two crushable ampoules said ampoules containing components of an active solvent comprising predetermined amounts of an apolar organic solvent, a polar organic solvent, an iron complexing agent, an organic acid, water, and a reducing agent;
(b) crushing the ampoules inside the receptacle and mixing the entire contents of the receptacle until the lubricant is completely dissolved to form a test solution inside the receptacle;
(c) allowing the test solution to react completely;
(d) attaching filtration means to an open end of said receptacle to filter fluid simultaneously as said fluid is removed from said receptacle; and
(e) allowing the test solution to separate into a top layer and a bottom layer, the bottom layer comprising an iron complex, and removing at least a portion of the bottom layer from the receptacle through the filtration means into a second receptacle adapted for optical absorbance measurement of the test solution by means of a differential spectrometer.

9. A method according to claim 8, wherein the receptacle is a soft transparent or semi-transparent plastic container.

10. A method according to claim 9, wherein the ampoules in the container are crushed by applying pressure to the container.

11. A method according to claim 8, wherein the second receptacle is a VIS cuvette.

12. A lubricant analysis test kit for determining the iron content of an in-use lubricant, which may be used on-site comprising:
(a) a receptacle adapted to be opened and closed on one end and being further adapted to prepare a test solution containing at least two crushable ampoules said ampoules containing components of an active solvent comprising predetermined amounts of an apolar organic solvent, a polar organic solvent, an iron complexing agent, an organic acid, water, and a reducing agent;
(b) a dispensing device adapted to dispense a predetermined amount of lubricant into said receptacle
(c) filtration means to filter fluid simultaneously as said fluid is removed from said receptacle, said filtration means adapted to be fitted to the end of said receptacle when said end is open;
(d) a second receptacle which adapted to contain fluid and to be placed in a means to measure absorbance in the visible range; and
(e) means to measure the absorbance at a selected frequency and a baseline frequency and to translate and display these measurements as ppm iron content.

13. An apparatus according to claim 12, wherein the receptacle is a soft transparent or semi-transparent plastic container.

14. An apparatus according to claim 12, wherein the filtration means comprises a PTFE disc filter having a 0.45 m pore diameter.

15. An apparatus according to claim 12, wherein the second receptacle is a VIS cuvette.

16. An apparatus according to claim 12, wherein the means to measure the absorbance is a spectrometer that allows measurements at two selected frequencies in the visible range.

17. An apparatus according to claim 12, wherein the means for absorbance measurements is a spectrometer which allows displaying the iron content of the lubricant in ppm units.

18. The method of claim 1, wherein the lubricant is cylinder drip oil.

19. The process of claim 1, wherein the apolar organic solvent is selected from the group consisting of iso- and normal alkanes from C₆ through C₁₆, ethers (ROR′) where R and R′ are a combination of C₁ through C₄.

20. The process of claim 1, wherein the polar organic solvent is selected from the group consisting of acetonitrile, acetone, alcohols from C₂ through C₆ and dimethylsulfoxide.
